(19) United States
(12) Patent Application Publication
(10) Pub. No.: US 2019/0209394 A1
(43) Pub. Date: Jul. 11, 2019

(54) Novel Reusable Swim Diaper

(71) Applicant: Lässig Holding GmbH, Babenhausen (DE)

(72) Inventor: Claudia Lässig, Aschaffenburg (DE)

(21) Appl. No.: 16/333,253

(22) PCT Filed: Aug. 25, 2017
(86) PCT No.: PCT/EP2017/071450

§ 371 Date: Mar. 14, 2019

(30) Foreign Application Priority Data
Sep. 15, 2016 (EP) 16189060.3

(51) Int. Cl. A61F 13/49; A61F 13/496; A61F 13/511

(52) U.S. Cl. CPC A61F 13/49006; A61F 2013/15195; A61F 13/511; A61F 13/496

(57) ABSTRACT

A reusable swim diaper (10) in the form of a panty, with elastic bands (12) around the waist and/or leg openings, the diaper includes the following: a) an outer layer (14), said outer layer consisting of an elastic knitted fabric or wovens; b) an inner layer (16), said inner layer consisting of an elastic knitted fabric or wovens from natural and/or synthetic fiber materials; c) and the inner layer (16) is a water vapor-permeable and water-absorbing inner layer.

### FIELD OF THE INVENTION

The invention relates to a reusable swim diaper in the form of a brief with an elastic termination in the waist and/or leg region.

### PRIOR ART

In recent decades, reusable fabric diapers consisting by way of example of cotton have been replaced by highly absorbent disposable diapers.

Some of the reasons for this are the ability of disposable diapers generally to absorb much more moisture than, for example, fabric diapers, greater ease of use, and the possibility of easy disposal after soiling.

However, neither reusable fabric diapers nor conventional disposable diapers are suitable for infants or toddlers participating in swimming or other water-based activities.

Highly absorbent disposable diapers, and reusable fabric diapers are by way of example too bulky and absorb too much water to allow swimming or participation in other water-based activities. The weight increase after absorption of large quantities of water can be considerable and additionally represents a safety risk, because it hinders flotation of an infant or toddler and in the worst case can even cause drowning.

Another factor in addition to potential safety risks is that absorbent disposable diapers and fabric diapers are often uncomfortable when they absorb large quantities of water.

Many parents therefore change children's diapers frequently during, for example, swimming or bathing. Products that have become popular here are swim diapers which do not consist of highly water-absorbent materials such as cotton.

The materials of said children's swim diapers must have a number of properties. Firstly, it is essential to use materials that are waterproof, i.e. prevent ingress of water from outside (e.g. pool-/seawater) into the diaper or saturation of the swim diaper therewith; it should be noted here, however, that leg and waist terminations in reusable swim diapers, and also any stitched seams that may be present, do not provide a completely waterproof seal, with the result that ingress of (small quantities of) water is still possible. At the same time, it is essential to use materials that transport water and liquids from the body, e.g. urine, outward, out of the swim diaper. The materials used should accordingly firstly be breathable and secondly exhibit water-vapor permeability. Higher-priced products should moreover also provide moisture management (i.e. wicking); this means that materials should be used that are not only waterproof but also water-vapor-permeable (breathability/water-vapor permeability), and preferably materials should be used which, although they are not actually waterproof, transport the moisture arising (e.g. sweat or urine) rapidly and actively away from the body (moisture management/wicking). The term wicking accordingly denotes an effect where moisture is spread over a maximized area of material in order to accelerate evaporation.

Water vapor has by way of example two possible ways of passing through waterproof membranes: firstly via what are known as microporous membranes. The term “microporous” means that the membrane has microscopically small holes that are sufficiently large to permit escape of water vapor molecules but at the same time are sufficiently small to prevent ingress of the larger water molecules. Examples of known microporous membranes are GoreTex® and eVENT®. Both are based on a stretched polytetrafluorethylene (ePTFE) membrane. The term “stretched” means that the pores are brought to the size that permits passage of water vapor.

The problem with those microporous membranes is that body oils, sweat, and also creams, are absorbed onto the surface of the membrane, and leave channels in the ePTFE through which moisture can pass into the products. In order to prevent that, the membranes can be covered with, for example, a thin polyurethane (PU) layer. This gives the membrane long-lasting impermeability. However, this type of PU layer at the same time restricts breathability because it absorbs and retains moisture—and therefore such products feel wet internally after exercise. Sweat condenses on the PU layer and is absorbed there, and is slowly forced outward.

Alternatively, what are known as closed-cell membranes can be used. In the case of these, the moisture accumulates on the internal side. The membrane swells, and the water vapor molecules are transported through the membrane—a type of osmosis principle. This functions with a small time delay. Closed-cell membranes are significantly more robust because they have no pores which can become enlarged (permeable) or blocked (loss of breathability). Most closed-cell membrane systems are made of polyester.

There are moreover also waterproof coatings. There are very many of these, and they are primarily (in some cases microporous) polyurethane coatings. In all cases there must be partial pressure gradient in order to permit passage of water vapor.

All non-waterproof materials also permit passage of water vapor molecules. However, there is the attendant problem that many of the materials absorb and store the water vapor. This is a decisive factor in determining the functionality of the product, which varies according to the period for which the material is then wet or the time required for it to become dry again. Cotton, for example, draws moisture rapidly away from the body, but does not allow said moisture to escape. Cotton stores up to 60% of its own weight of moisture. That is disadvantageous for two reasons: firstly, the moisture rapidly cools the body, and secondly even if the membrane is breathable it cannot function if the layer(s) thereunder store(s) the moisture, which therefore cannot escape. Alternative materials are the synthetic polymer materials, the moisture absorption of which at standard temperature (20° C.) and 65% humidity, based on their own weight, is about 7% (acrylic; (AC)), 0.5% for polyester (PES), 4.5% for polyamide (PA) and 0% for polypropylenes (PP). Because there is a noticeable functional difference even between 0% and 7% moisture absorption, materials considered to be breathable are PES and PP.

Certain knitted structures with various levels can conduct moisture outward by mechanical means. Twin-ply materials, i.e. materials consisting of two components with different properties, have higher activity in outward dissipation of moisture. The moisture is then sometimes still detectable on the external ply, while internally the material feels dry. An important factor for good wicking materials is rapid distribution of the moisture over a maximized area. The moisture is thus better able to evaporate, or can be passed onward over a larger area to the next layer.

All of these properties of materials must be ideally combined with one another in order to meet the requirements of the parents and of the children in reusable swim diapers.

U.S. Pat. No. 7,678,094B1 discloses a multiple-ply reusable swim diaper with individual plies having certain properties such as breathability, water-vapor permeability, etc. The swim diaper described has the disadvantage that, for example, the individual plies are held together by stitched seams; this can lead to friction on the very sensitive skin of children, with possible resultant reddening and swelling. Furthermore, although the ply facing toward the skin uses materials with a wicking effect, these are not the unidirectional-wicking material combinations as in the present invention which minimize moisture contact with the skin and thus provide a pleasant and skin-friendly feel.

EP 15176716.7 discloses a multiple-ply reusable swim diaper which is composed of at least three plies (external layer, intermediate layer and internal layer). The intermediate layer is in essence present in the crotch region of the swim diaper between the external and internal layer, and is intended to ensure absorption of excess liquids/water. The disadvantage of such multiple-ply swim diapers with intermediate layer content (“patch constructions”) consists inter alia in concentration of the accumulated moisture in the relatively small-area patch in the lower crotch region. This tends to cause downward movement of the swim diaper once the intermediate layer/patch has been filled with liquid. This results in separation of the moisture storage system from the skin, inhibiting evaporation (vaporization) of the liquid on the skin.

An Internet article (Anonymous: “Because swim diapers are in the water they don't retain urine!”—The test on Totsbots swim diapers) discloses simple diapers which are not configured as a brief and comprise microfiber toweling (internal layer), which becomes saturated with water. As may be concluded simply from the title (Because swim diapers are in the water they don't retain urine!), there is no water-vapor permeability provided because the purpose for which said diapers are designed is not to retain any liquids in the diaper but only to retain solid faeces and thus isolate these from the surrounding medium (water).

US 2002/0010452 A1 describes a swim diaper which is likewise composed of a plurality of plies, but at least three plies, and has inter alia an internal layer and an intermediate layer, where the internal layer passes moisture onward to the intermediate layer, i.e. to an inlay, where said moisture is absorbed. Again, that document merely describes the known, three-ply swim diaper (with, between internal layer and external layer, an inlay which absorbs the liquid).

The same also applies to EP 2 918 253 A1 and to the abovementioned U.S. Pat. No. 7,678,094 B1, which likewise describe at least three-ply swim diapers. Between the internal layer and external layer there is an inlay (intermediate layer), which is water-absorbent and absorbs the moisture passed onward from the internal layer.

### Object

In the light of the above, it is an object of the present invention to provide a reusable swim diaper which exhibits high wearer comfort and at the same time keeps skin and environment very substantially moisture-free and thus extends the field of use of such swim diapers. Another object of the invention consists in providing a reusable swim diaper which differs from the known prior art in that it has no intermediate layer and thus inter alia can provide greater wearer comfort at the same time as improved transport of moisture away from the skin.

### Achievement of Object

Said object is achieved by the invention with the features of the independent claim. Advantageous further developments of the invention are characterized in the dependent claims. The wording of all of the claims is hereby incorporated by reference into the content of this description. The invention also comprises all of the useful combinations of independent and/or dependent claims, and in particular all of such combinations that are mentioned.

A reusable swim diaper of the invention is designed in the form of a brief with an elastic termination in the waist and/or leg region. The swim diaper moreover comprises:

a) an external layer, where the external layer has been constructed from an elastic knitted fabric or a woven fabric;

b) an internal layer, where the internal layer has been constructed from a knitted fabric or a woven fabric made of natural and/or synthetic fiber materials;

c) and where the internal layer is a water-vapor-permeable and water-absorbent internal layer.

In this connection it is important to understand that the internal layer of the invention must be bifunctional. The desired properties of the internal layer, namely water-vapor-permeability together with water-absorbency, are normally mutually exclusive. In the case of conventional swim diapers, an (water-absorbent) intermediate layer is applied to the internal layer and absorbs the moisture passed onward by the internal (water-vapor-permeable) layer. The swim diaper of the invention, however, omits said intermediate layer entirely; a novel diaper structure is thus required.

The particular feature of this novel diaper structure consists inter alia of a “patchless” or intermediate-layer-free internal structure which allows moisture arising internally, i.e. toward the skin, to be spread over a storage and evaporation region that is many times larger than in the case of conventional swim diaper structures with patch. This produces a more pleasant feel for the wearer, and more freedom of movement. Another consequence of the large-area distribution is increased evaporation rate, which is achieved not only through the greater area distribution but also by the fact that the moisture storage system (water-absorbent internal layer) is not located—as in the case of the conventional patch structures—in the crotch region. This spacing of the moisture reservoir in conventional swim diapers inhibits rapid evaporation, because rapid vaporization requires body heat which allows the moisture to vaporize from the internal region (skin) toward the external region (environment). From this it follows that increasing proximity of the moisture to the external layer results in more evaporation caused by the correspondingly accumulating body heat.

In the case of the conventional swim diaper, however, the evaporation region corresponds precisely to the region where the distance between the moisture and the heat source (body) is greatest.

The size and arrangement of the moisture storage system in the swim diaper of the invention, however, achieves evaporation that is many times better over the entire extent of the diaper, because moisture transfer here initially takes place into the storage system (unidirectional wicking). By virtue of the structure of the storage system in fiber form, moisture transport takes place rapidly and preferably within the storage-system fibers. The moisture is then transported over a wide area and into the entire extent of the diaper, i.e. by way of example also into the back and abdominal region, which can achieve a higher evaporation rate because of greater closeness to the body; (the unidirectional wicking however prevents exposure of the skin in said regions to increased contact with moisture).

The swim diaper of the invention differs from other swim diaper structures in having a moisture reservoir that is many times larger. Said reservoir can absorb any moisture arising until evaporation has taken place. Other diaper structures usually lead to premature escape of the moisture through, for example, stitched seams or through leg terminations, because in the absence of an adequate storage-system component the quantity of moisture rapidly arising does not experience any corresponding moisture absorption.

The expression “elastic termination in the waist and/or leg region” comprises all of the terminations known to the person skilled in the art, for example elastic bands, strands, tapes, filaments, filament beads, tensioning systems and the like, these being arranged close to an opening of the reusable swim diaper into which a leg and/or a waist of a wearer is inserted. An example of a preferred elastic termination is a rubber band.

Said elastic termination can be attached to the reusable swim diaper in the waist and/or leg region via one or more stitched seams, for example two stitched seams.

The invention also comprises alternative terminations. It is therefore also possible by way of example to use buttons, zip fastenings or touch-and-close fastenings and the like.

The swim diaper of the invention has an external layer, where the external layer has been constructed from an elastic knitted fabric or a woven fabric.

The expression “elastic knitted fabric” comprises all of the textile sheet materials known to the person skilled in the art in which a loop formed by filaments has been interlocked into another loop. The knitted fabrics thus produced can be formed with use of one or more filaments. The formed-loop knitted fabric and drawn-loop knitted fabric are by way of example among the knitted fabrics and are comprised in the invention.

The advantage of formed-loop knitted materials consists in their great elasticity. Formed-loop knits therefore have excellent suitability for the swim diaper of the invention. Wool and coarse yarns are suitable for formed-loop knitting, and very fine yarns are equally suitable. Examples of elastic knitted fabrics of the invention are elastic knitted fabrics made of polyamide, polypropylene, polyester or appropriate mixtures of these materials, elastane (block copolymer of the constituents polyurethane and polyethyleneglycol) and other elastic fibers and mixtures thereof, formed-loop nylon knits and formed-loop knits produced with use of elastic fibers such as piqué, fleece and panne velvet.

The expression “woven fabric” comprises all of the weaving products known to the person skilled in the art, manufactured manually or by machinery, for example cloth, velvet, velour, plush, toweling and other textile sheet materials made of at least two filament systems crossed at right angles or almost at right angles.

Woven fabrics are particularly strong and robust when they have high filament densities. This effect is further amplified when threads are used instead of simple yarns.

Types of woven fabric(s) comprised in the invention can by way of example be: cambric, brocade, chiffon, chintz, crepe, enoa, damast, denim, drill, etamine (sieve cloth), end-on-end, flannel, gabardine, georgette, jersey, loden, natte, cheesecloth, pinpoint, piqué, poplin, satin, seersucker, taffeta, cloth, tweed, two-ply, broadcloth.

In a preferred embodiment, the water-vapor-permeable and water-absorbent internal layer features a formed-loop knit or a textile construct or woven fabric. It is preferable that said formed-loop knit or said textile construct or said woven fabric consists of identical or different yarns and/or threads.

The expression “yarns or threads” of the invention means any of the natural and synthetic (chemical) fibers that are processed to give yarns and threads.

Yarn in the sense of “single yarn” here is a single-filament textile construct which consists of spun fibers or of filaments (continuous fibers). A spun-fiber yarn generally consists of a number of spun fibers held together by twisting (spinning). A filament yarn generally consists of one or more filaments produced with or without twisting. A multiple-wound yarn consists of two or more yarns or threads which are wound together but not twisted with one another.

Thread is a collective term for all linear textile constructs produced by twisting together of simple yarns and/or threads of identical or different type. Single-stage threads are produced in a twisting procedure from two or more simple yarns. Multistage threads are produced in one or more additional twisting procedures from single-stage and/or multistage threads, optionally also with concomitant use of simple yarns. The twisting increases the strength of the filament, and improves uniformity. Threads are stronger and more robust than yarns of identical fineness. Appropriate modification of processing can provide pattern effects to threads. The best-known threads of this type are toweling thread, flame thread, slub thread, crimp thread, loop thread and caterpillar thread. The word “thread” in names of materials indicates a particularly strong and durable material consisting of twisted yarns. The invention comprises all yarns and/or threads, and also mixtures thereof.

In another preferred embodiment, a feature of the reusable swim diaper of the invention is that the internal layer taking the form of formed-loop knit, textile construct and/or woven fabric is configured as single drawn-loop knit, multiple drawn-loop knit and/or sheet material. The formed-loop knits, textile constructs and/or woven fabrics here can consist of identical or different yarns and/or threads. This can be the case either within one level of material or on the various material sides (for example upper side and underside), as required by the properties that the material is intended to have. It is preferable that the sides of the formed-loop knit or of the textile construct or of the woven fabrics consist of different yarns and/or threads. It is therefore possible by way of example that the material side facing toward the skin is composed of yarns and/or threads different from those in the material side facing away from the skin (facing toward the environment).

The expression “multiple structure” in the invention means a structure consisting of a plurality of layers which are produced simultaneously and which are bonded to one another via the structure itself (e.g. Jacquard) or else via additional processing steps. Multiple structures are always durably bonded materials which can provide cavities but which in themselves form a coherent ply, because the components cannot be separated from one another without destruction of the entire structure. An example of such multiple structures comprised by the invention is a double drawn-loop knit where two mutually superposed knitting looms respectively produce one ply, and these have been bonded by perpendicular filaments which themselves are a fixed component of both plies and contribute to the single-ply structure. It is possible in the invention that absorption fibers are inserted into the resultant cavities and, by virtue of their increased absorbency provide the necessary storage capacity, but also the durable moisture extraction from the material layer on the skin side. The person skilled in the art is aware of other single drawn-loop knits, multiple drawn-loop knits and/or sheet materials, and these are likewise comprised by the invention.

In another preferred embodiment, a feature of the swim diaper of the invention is that the formed-loop knit, textile construct and/or woven fabric comprises spacing elements, spacers and/or fiber chambers.

It is preferable that the spacing elements and/or spacers are drawn-loop spacer knits, formed-loop spacer knits and/or spacer yarns.

Drawn-loop spacer knits generally consist of two webs bonded to one another by a spacer filament. They are used by way of example for the production of items of sports apparel such as tracksuits or heat-retention suits, and also of inserts for the sole or sidewall-region of shoes.

In a known method for influencing the extensibility of drawn-loop plain knits, in particular in connection with plush fabrics, at least one additional floating or plating filament is incorporated into selected courses of a drawn-loop knit base, and is processed in part to give stitches and in part to give floats. However, drawn-loop spacer knits are not produced on plush machines, but instead are generally produced on circular border knitting machines, where one of the webs is knitted by the needles of a needle cylinder and the other web is knitted by the needles of a rib dial. Knitting machines of this type where the needles of the cylinder and of the rib dial are staggered are also called fine rib machines, at least when they have relatively small needle spacings and large diameter. A disadvantage of such knitting machines is that the two webs must respectively be produced with all of the needles of one or other needle bed, and transverse elasticity and transverse extension, and also other properties, can therefore be altered only via the selection of the yarn used, and only within narrow limits.

The expression “spacer structure” (or Spaceyarn construction) means in essence a structure in which a plurality of parallel layers are bonded by one or more spaceyarns, and space is generated here between the plies either via the structure itself or else via a required stiffness of the spaceyarn. Advantages of such a structure are inter alia that various materials with different properties—for example in relation to their structure and also composition—can be bonded to one another. It is thus possible to achieve bonding between, for example, metal yarn structures externally (cold, hard, shape-determining, waterproof, etc.) and velvet fabric internally (soft, conformable shape, warm, water-absorbent, etc.).

A spacer structure is a type of multiple structure. The invention provides use of relatively stiff spacer filaments, or else no use of spacer filaments but use of a pile filament to bond the individual layers to one another, and filling of the functional cavity by various types of knitted external and internal plies with different physical properties (elasticity, etc.). There can moreover also be moisture-storing spacer fibers/bulked filaments between the layers. Other structures included in the invention comprise spacer structures or a two- or multicomponent layer comprising no spacer fibers/bulked filaments. In general terms, the invention comprises all of the structures known to the person skilled in the art that comprise spacing elements, spacers and/or fiber chambers.

Multiple structures or formed-loop spacer knits are double-ply textiles in which the warp-knitted plies are held apart by filaments known as pile filaments which have spacer and bonding functions. The formed-loop spacer knits are knitted fabrics or formed-loop knits which have been extended into the third dimension.

A drawn-loop spacer knit or formed-loop spacer knit has, by virtue of its thickness, good properties in respect of softness, moisture transport, and also moisture storage. It is preferable to adjust the thickness of the drawn-loop spacer knit or formed-loop spacer knit in accordance with the loads to be expected during the wearing of the swim diaper. By way of example, the thickness of the drawn-loop spacer knit or formed-loop spacer knit could be greater in the crotch region than in the back region, in order to achieve targeted reduction of exposure to forces and to friction in the crotch. The thickness of the drawn-loop spacer knit can vary throughout the swim diaper, and this could also be present only in a prescribed region.

It is moreover preferable that the plies of the drawn-loop spacer knit or formed-loop spacer knit comprise different yarns or threads. The drawn-loop/formed-loop spacer knit can thus fulfill different functions within the swim diaper. By way of example, the side facing toward the skin can comprise moisture-absorbing yarn and/or thread, the side facing away from the skin can comprise rubbery yarn and/or thread, and the yarn and/or the thread between these sides, i.e. the spacer yarn, can be stable nylon yarn. The invention moreover comprises use of a voluminous and/or hollow yarn or thread which can absorb impact forces, and also use of a moisture-absorbing yarn and/or thread.

In this connection it is important to point out that the swim diaper of the invention or the inner layer of the invention has the following functions.

a. It features a specific moisture-management function (unidirectional wicking). This function has the effect that the liquid encountering the internal side of the diaper is immediately transported to another component of the material of the internal layer, which serves as moisture storage system, and which therefore extracts the moisture on the internal side facing toward the body.

b. It features a moisture storage system (absorbing component). The moisture here is stored temporarily until evaporation takes place on the external side of the diaper. The design of the reusable swim diaper of the invention can achieve the increased water-absorption capacity inter alia by using storage fibers, and also specific yarn structures, for example bulked yarns which give preference to preferred transport of moisture along and within storage-fiber chambers, rather than to onward passage of said moisture to the external layers. The orientation of these absorption chambers within the swim diaper of the invention can be such as to direct transport of moisture within and along the fiber channels, so that during the wearing of the swim diaper the moisture can pass into the regions where it is most likely to vaporize.

c. The material side of the internal layer that is remote from the body/remote from the skin is equipped with quick-dry properties which are responsible for rapid escape of the moisture by vaporization through the covering material or external layer. The stored moisture can therefore immediately spread over a substantial area in order to vaporize (wicking/quick-dry properties).

The functions described can be achieved by virtue of a specific structure which derives from knitted-fabric or woven-fabric technology and consists of a complex of various fiber components with different hydrophilic/hydrophobic properties. An example of a material suitable for this purpose is a knitted polyester fabric.

The complex functional process is moreover completed by surface treatments carried out on the finished material. By virtue of the inventive combination of conventional quick-dry properties with the unidirectional wicking finish intrinsic to the invention, the reusable swim diaper is therefore a unique product, since contact of moisture with the skin is reduced to a minimum. This contrasts with commercially available swim diapers in which the simple wicking function produces precisely the opposite condition: wicking properties maximize spread of the moisture, i.e. body contact with the moisture is maximized. This contact with moisture is not only unpleasant and unfriendly to the skin but is further exacerbated by the waterproof properties of the adjacent external material of conventional swim diapers; said properties considerably inhibit, or prevent, transport of moisture via vaporization.

In an alternative, the internal layer has not been manufactured in a single piece as spacer knit, but instead has been manufactured separately (e.g. drawn-loop-knitted) and then assembled (e.g. stitched).

It is preferable to fill intervening spaces in the spacer knit with soft materials, in order to achieve additional functions. By way of example, the intervening spaces could be filled with fibers, yarns (spacer yarns), chambers (fiber chambers) and/or additional fibers.

It is further preferable that said materials are replaceable, so that the user can adjust the properties to meet requirements. By way of example, the knitted fabric in the crotch region could have been drawn-loop-knitted in a manner such that it has openings, pockets and/or tunnels which hold the materials in a manner that permits replacement.

In another preferred embodiment, a feature of the reusable swim diaper is that the formed-loop knits, textile constructs and/or woven fabrics have in essence been bonded to one another without stitched seam. This can be achieved by way of example by means of welding (pressure welding), preferably ultrasonic welding, adhesive bonding and/or fusion, thus ensuring achievement of a durable bond.

Ultrasonic welding is an industrial technique in which high-frequency ultrasound is applied locally in order to hold workpieces together under pressure and thus to create a solid body (welding). It is in general use for the synthetic polymers used, and in particular for various bonding materials.

In another particularly preferred embodiment, a feature of the reusable swim diaper is that the water-vapor-permeable and water-absorbent internal layer consists of a knitted fabric or a woven fabric made of synthetic fiber materials, preferably an internal polyester layer, in particular a polycarbonate (PC), polyethylene terephthalate (PET) and/or polyester resin internal layer.

The term “polyesters” in the invention means any of the polymers having ester functions —[—CO—O—]— in their main chain. These include by way of example the synthetic polymers, among which are the widely used polycarbonates (PC), polyethylene terephthalate (PET), and unsaturated polyester resin (UP), which is a thermoset. Examples of other fibers made of synthetic polymers which are comprised in the invention are polyamides (PA), polyimides (PI), polyamideimides (PAI), polyphenylene sulfides (PPS), aramids, polyacrylonitriles (PAN), polytetrafluoroethylenes (PTFE), polyethylenes (PE), polypropylenes (PP), polyvinyl chlorides (PVC or in the case of fibers CLF) and polyurethanes (EL).

The water-vapor-permeable and water-absorbent internal layer is particularly preferably composed of materials which have unidirectional-wicking properties. The meaning of this in the invention is a further development of conventional wicking in which the moisture content of the material is controlled not only in relation to magnitude but also in relation to position. This means that the moisture encountering the upper side of the material is initially rapidly drawn to the underside of the material, from where the wicking function (=maximized distribution of the moisture) then operates. It follows from the above that only small quantities of residual moisture remain on the upper side of the material (this being the side facing toward the body in the case of the swim diaper of the invention), whereas most of the moisture is drawn away to the side facing away from the body, and vaporizes there. In contrast to the above, in the case of conventional wicking the moisture content becomes uniformly distributed across a large area on the upper side and underside of the material; this can lead to correspondingly “maximized and large-area” contact of the (sensitive) child's (toddler's) skin with the accumulated moisture and to skin irritation, reddening, friction, reduced wearer comfort, etc. Examples of materials that have appropriate unidirectional-wicking properties and accordingly can be used as water-vapor-permeable internal layer are the synthetic fiber materials described, in particular the polyester fiber materials. The person skilled in the art is aware of other synthetic fiber mixtures which can be used in the invention.

In another preferred embodiment, the water-absorbent components of the internal layer are an internal layer component made of toweling and/or made of fleece. Toweling has the advantage of particular absorbency together with pleasant hand. It is preferable that the water-absorbent internal-layer component is in essence distributed across the entire swim diaper, i.e. applied across the entire internal layer.

In a preferred embodiment, the external layer moreover consists of waterproof materials or a mixture of waterproof and water-permeable materials.

In another preferred embodiment, a feature of the reusable swim diaper is that the external layer has a coating and/or a membrane, preferably a microporous, hydrophobic and/or hydrophilic membrane. This coating and/or membrane can have been bonded over a large area to other layers. These other layers can likewise be textiles, foils made of synthetic polymer or of metal, foam, or other layers. The bonding is achieved coherently, for example via adhesive bonding or fusion in specific textile machines (lamination). The coating and/or the membrane is in particular intended to ensure, or increase, the level of waterproofing provided by the reusable swim diaper.

The lamination produces a material which combines the properties of its starting materials: By way of example, a strong weave provides tear resistance and abrasion resistance, a synthetic polymer foil provides waterproof and windproof properties, and the combination with a metal foil gives an opaque material which in some circumstances also reflects radiated heat. The external layer can by way of example have been laminated to a very thin membrane foil that is windproof, breathable and/or waterproof.

The coatings used can be either fiber coatings or large-area coatings. If, by way of example, Teflon is used as coating (and not as membrane), the fibers are sheathed with the synthetic polymer.

In the case of a large-area coating, which is mostly applied to a supporting material from a vapor or by spreading, it is possible in the invention to use simple or microporous coatings. In the case of the simple variant (waterproof but not breathable), a coherent coating is applied to the surface of the woven fabric; ingress of water is no longer possible, and no moisture escapes.

In the case of microporous membranes (waterproof and breathable), there are tiny pores passing through the coating film which prevent ingress of moisture but permit escape of water vapor. In the case of these coatings, breathability is retained. The same characteristics are exhibited by hydrophilic and hydrophobic membranes.

There are various possibilities for the processing of membranes on textiles: In the case of an overlay laminate, the membrane is bonded directly to the overlay to give a two-ply laminate. In the case of an insert laminate, the membrane is laminated onto a lightweight supportive material and processed between overlay and lining. In the case of a lining laminate, the membrane is bonded on the reverse side of the lining material. In the case of a three-layer laminate, the membrane is bonded not only to the overlay but also to the lining material.

The invention also comprises all of the processing methods described and known to the person skilled in the art for the application of a coating and/or of a membrane on textiles.

An example of a coating preferred in the invention is a synthetic polymer coating and/or synthetic resin coating, for example a polyurethane (PU) or polyvinyl chloride (PVC) coating. The PU coatings have the advantage that they are less heavy than PVC coatings, and also remain elastic at low temperatures, and are more environmentally compatible.

In another preferred embodiment, the membrane is preferably a microporous membrane, in particular a synthetic and/or semisynthetic polymer membrane, preferably a polytetrafluoroethylene (PTFE) membrane, in particular a stretched polytetrafluoroethylene (PTFE) membrane.

Another feature of the reusable swim diaper of the invention is inter alia that the coating and/or (microporous) membrane has in essence been applied to the external layer without stitched seam. A process for producing such bonds without stitched seam is by way of example that known as welding (pressure welding). Alternative processes are by way of example the adhesive bonding/fusion described above, in specific textile machines. A feature of a particularly preferred reusable swim diaper is therefore that the coating and/or (microporous) membrane has been durably applied to the external layer by means of welding (pressure welding). In general terms, it should be noted that between external layer, coating and/or (microporous) membrane there is, as far as possible, a stitched-seam-free bond present, because a stitched seam requires perforation and therefore restricts waterproof properties.

In addition to the above, each external layer can also have a sun-protection factor, more precisely a UV-protection factor (UPF). The UPF of the external layer is preferably >15, particularly preferably >40. The sun-protection property is provided to the external layer by means of introduction or application of UV-absorbing or -reflecting agents, via use of particularly densely woven materials, or via careful selection of color.

In another preferred embodiment, a feature of the reusable swim diaper is that the coating and/or microporous membrane has in essence been applied to the external layer without stitched seam. Preferred processes for the application of the coating and/or of a microporous membrane without stitched seam are by way of example welding (pressure welding), adhesive bonding and/or fusion, with resultant achievement of a durable bond to the external layer.

Further details and features are apparent from the description below of preferred inventive examples in conjunction with the dependent claims. The respective features here can be realized per se alone or in groups in combination with one another. The possibilities for achieving the object are not restricted to the inventive examples: By way of example, all stated ranges always comprise intermediate values—that are not mentioned—and all conceivable subintervals.

The inventive examples are depicted diagrammatically in the figures. Identical reference signs in the individual figures here denote identical elements or elements that are functionally identical or that correspond to one another in respect of their functions. Details are as follows:

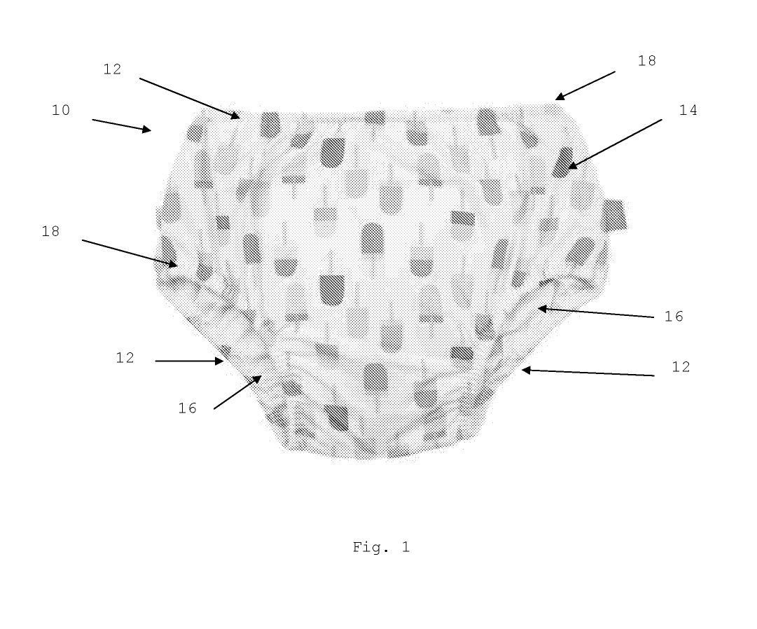

FIG. 1 depicts one embodiment of the reusable diaper;

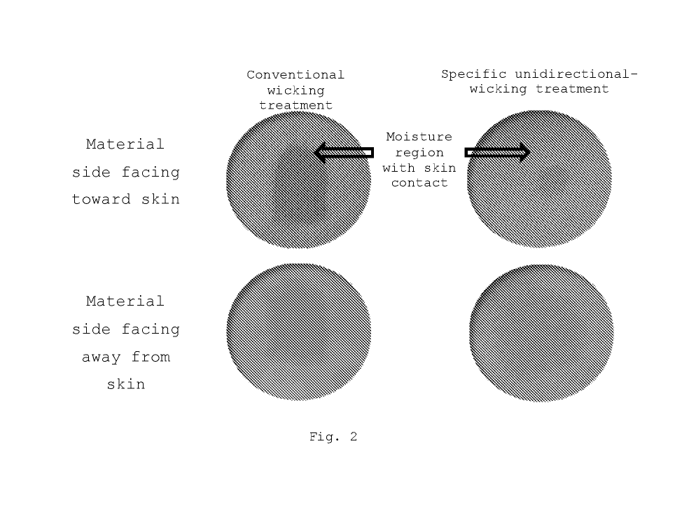

FIG. 2 compares materials with normal wicking and unidirectional-wicking properties;

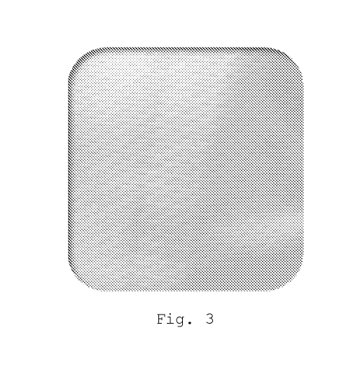

FIG. 3 depicts a welded toweling layer;

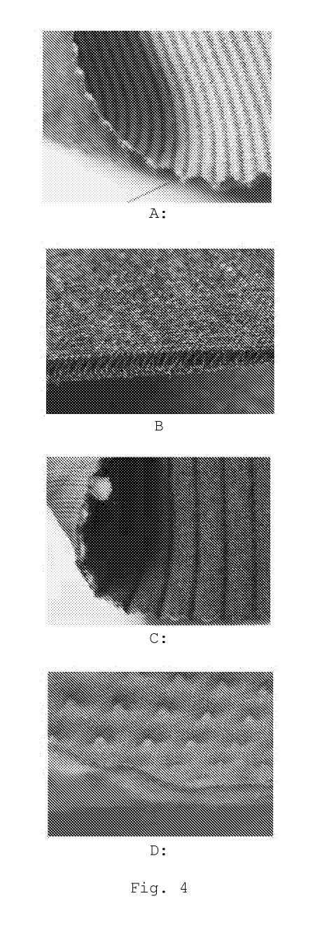

FIG. 4 depicts various embodiments of the internal layer of the reusable swim diaper;

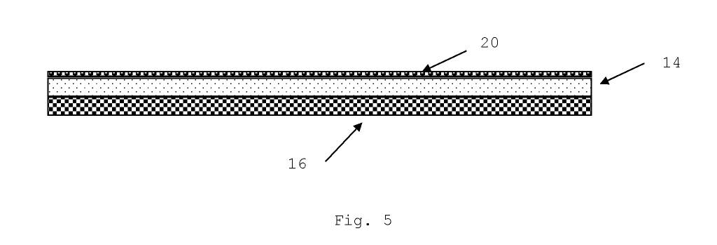

FIG. 5 is a diagram of the layer structure of one embodiment of the reusable swim diaper.

FIG. 1 shows an embodiment of the reusable swim diaper (10) in the form of a brief with an elastic termination, in the present case made of elastic, in the waist and/or leg region (12), and also an external layer (14) and an internal layer (16). FIG. 1 moreover shows that the elastic termination has been secured to the reusable swim diaper (10) by means of a stitched seam (18).

FIG. 2 compares materials with normal wicking properties and unidirectional-wicking properties. It can clearly be seen that in the case of the unidirectional-wicking materials the moisture arising on the upper side of the fabric is first rapidly drawn to the underside of the fabric, from where the wicking function (=maximized moisture distribution) then operates. It therefore follows that only small quantities of residual moisture remain on the upper side of the material (this being the material side facing toward the body in the case of the swim diaper of the invention), while most of the moisture is drawn away to the side facing away from the body, and evaporates there. In contrast thereto, in the case of conventional wicking the moisture on the upper side and underside of the fabric is distributed uniformly over a large area; this leads to maximized contact of the skin with the accumulated moisture over a substantial area.

FIG. 3 shows an additional moisture-storage system in the form of a welded toweling material. This can be used as internal layer (components) in the invention, where—as set out at an earlier stage above—the bonding of the internal layer to the external layer can be achieved in the present case by means of welding (pressure welding) without stitched seam.

FIG. 4 shows the structure of various internal layers of the invention: 4A: Internal layer configured as single drawn-loop knit with various yarn components on both material sides; 4B: Internal layer configured as multiple drawn-loop knit with spacer yarns as spacer; 4C: Internal layer configured as multiple drawn-loop knit with fiber chambers; 4D: Internal layer configured as composite sheet material in which two functional components have been durably bonded to one another by means of large-area ultrasonic welding.

FIG. 5 is a diagram of the layer structure of the reusable swim diaper, and shows the external layer (14), the internal layer (16), and also a coating (20) applied to the external layer (14) and intended for reliable provision of waterproof properties. Said figure also shows that the internal layer (16) and external layer (14) have been secured to one another without stitched seam. This can by way of example be achieved by means of welding (pressure welding), adhesive bonding or fusion in specific textile machines. The coating (20) has likewise been applied to the external layer (14) without stitched seam. The bond is achieved here in coherent manner, for example via adhesive bonding or fusion in specific textile machines (lamination) by means of welding (pressure welding).

There are numerous realizable modifications and further developments of the embodiments described.

### KEY

10 Reusable swim diaper
12 Elastic termination
14 External layer
16 Internal layer
18 Stitched seam
20 Coating

### REFERENCES

References
U.S. Pat. No. 7,678,094B1
EP 15176716.7
US 2002/0010452 A1
EP 2 918 253 A1

### Non-Patent References

Anonymous: “Because swim diapers are in the water, they do not retain urine!—Test report on Totsbots swim diapers” (published: 13 May 2016)

1. A reusable swim diaper in the form of a brief with an elastic termination in the waist and/or leg region, comprising:
a) an external layer, where the external layer comprises an elastic knitted fabric or a woven fabric;
b) an internal layer, where the internal layer comprises a knitted fabric or a woven fabric made of natural and/or synthetic fiber materials;
c) and where the internal layer is a water-vapor-permeable and water-absorbent internal layer.

2. The reusable swim diaper as claimed in claim 1, wherein the water-vapor-permeable and water-absorbent internal layer is a formed-loop knit or a textile construct.

3. The reusable swim diaper as claimed in claim 2, wherein the formed-loop knit or the textile construct consists of identical or different yarns and/or threads.

4. The reusable swim diaper as claimed in claim 2, wherein the formed-loop knit or the textile construct is configured as single drawn-loop knit, multiple drawn-loop knit and/or sheet material.

5. The reusable swim diaper as claimed in claim 2, wherein the material sides of the formed-loop knit or of the textile construct comprise different yarns and/or threads.

6. The reusable swim diaper as claimed in claim 2, wherein the formed-loop knit or the textile construct comprises spacing elements, spacers and/or fiber chambers.

7. The reusable swim diaper as claimed in claim 6, wherein the spacing elements and/or spacers are drawn-loop spacer knits, formed-loop spacer knits and/or spacer yarns.

8. The reusable swim diaper as claimed in claim 2, wherein formed-loop knits or textile constructs are bonded to one another without stitched seam.

9. The reusable swim diaper as claimed in claim 8, wherein the formed-loop knits or textile constructs are bonded durably to one another by welding.

10. The reusable swim diaper as claimed in claim 1, wherein the internal layer made of a knitted fabric or of a woven fabric made of synthetic fiber materials is an internal polyester layer.

11. The reusable swim diaper as claimed in claim 1, wherein the external layer has a waterproof coating and/or a microporous membrane.

12. The reusable swim diaper as claimed in claim 11, wherein the coating is a synthetic polymer coating and/or synthetic resin coating.

13. The reusable swim diaper as claimed in claim 11, wherein the microporous membrane is a synthetic and/or semisynthetic polymer membrane.

14. The reusable swim diaper as claimed in claim 11, wherein the coating and/or microporous membrane is applied to the external layer without stitched seam.

15. The reusable swim diaper as claimed in claim 14, wherein the coating and/or microporous membrane is durably applied to the external layer by welding, adhesive bonding and/or fusion.

16. The reusable swim diaper as claimed in claim 8, wherein the formed-loop knits or textile constructs are bonded durably to one another by ultrasonic welding, adhesive bonding and/or fusion.

17. The reusable swim diaper as claimed in claim 1, wherein the internal layer made of a knitted fabric or of a woven fabric made of synthetic fiber materials is a polycarbonate, polyethylene terephthalate and/or polyester resin.

18. The reusable swim diaper as claimed in claim 11, wherein the coating is a polyurethane coating.

19. The reusable swim diaper as claimed in claim 11, wherein the microporous membrane is a polytetrafluoroethylene membrane.
